(19) United States
(12) Patent Application Publication
(10) Pub. No.: US 2002/0006671 A1
(43) Pub. Date: Jan. 17, 2002

(54) Luminescence polarization assays

(75) Inventors: J. Richard Sportsman, Palo Alto, CA (US); Lawrence M. Kauvar, San Francisco, CA (US)

Correspondence Address: James R. Abney, Suite 200, 520 S.W. Yamhill Street, Portland, OR, 97204, US

(73) Assignee: LJL BioSystems, Inc.

(21) Appl. No.: 09/770,724

(22) Filed: Jan. 25, 2001

Related U.S. Application Data
(63) Continuation of application No. PCT/US00/15774, filed on Jun. 9, 2000.
(60) Provisional application No. 60/138,311, filed on Jun. 9, 1999.

(51) Int. Cl. G01N 33/53; C07K 1/04; G01N 33/543

(52) U.S. Cl. 436/518; 435/7.1; 530/334

(57) ABSTRACT

Improvements in luminescence polarization assays. The improvements may include using confocal optics to increase sensitivity and accuracy, providing additional classes of tracers, providing improved methods to prepare specific luminescent tracers, and/or expanding the scope of assay targets, among others.

### CROSS-REFERENCES TO RELATED APPLICATIONS

This application is a continuation of PCT Patent Application Ser. No. PCT/US00/15774, filed Jun. 9, 2000, which is incorporated herein by reference.

This application is based upon and claims priority under 35 U.S.C. §119 from the following U.S. Provisional Patent Application, which is incorporated herein by reference: Ser. No. 60/138,311, filed Jun. 9, 1999.

This application incorporates by reference the following U.S. patent applications: Ser. No. 08/840,553, filed Apr. 14, 1997; Ser. No. 08/929,095, filed Sep. 15, 1997; Ser. No. 09/118,141, filed Jul. 16, 1998; Ser. No. 09/144,575, filed Aug. 31, 1998; Ser. No. 09/144,578, filed Aug. 31, 1998; Ser. No. 09/146,081, filed Sep. 2, 1998; Ser. No. 09/156,318, filed Sep. 18, 1998; Ser. No. 09/160,533, filed Sep. 24, 1998; Ser. No. 09/302,158, filed Apr. 29, 1999; Ser. No. 09/349,733, filed Jul. 8, 1999; Ser. No. 09/468,440, filed Dec. 21, 1999; Ser. No. 09/478,819, filed Jan. 5, 2000; Ser. No. 09/494,407, filed Jan. 28, 2000; and Ser. No. 09/556,030, filed Apr. 20, 2000.

This application also incorporates by reference the following PCT patent applications: Ser. No. PCT/US99/01656, filed Jan. 25, 1999; Ser. No. PCT/US99/03678, filed Feb. 19, 1999; Ser. No. PCT/US99/08410, filed Apr. 16, 1999; Ser. No. PCT/US99/16057, filed Jul. 15, 1999; Ser. No. PCT/US99/16453, filed Jul. 21, 1999; Ser. No. PCT/US99/16621, filed Jul. 23, 1999; Ser. No. PCT/US99/16286, filed Jul. 26, 1999; Ser. No. PCT/US99/16287, filed Jul. 26, 1999; Ser. No. PCT/US99/24707, filed Oct. 19, 1999; Ser. No. PCT/US00/00895, filed Jan. 14, 2000; Ser. No. PCT/US00/03589, filed Feb. 11, 2000; Ser. No. PCT/US00/04543, filed Feb. 22, 2000; Ser. No. PCT/US00/06841, filed Mar. 15, 2000; Ser. No. PCT/US00/12277, filed May 3, 2000; Ser. No. PCT/US00/16025, filed Jun. 9, 2000; and Ser. No. PCT/US00/16012, filed Jun. 9, 2000.

This application also incorporates by reference the following U.S. provisional patent applications: Ser. No. 60/138,438, filed Jun. 10, 1999; Ser. No. 60/138,737, filed Jun. 11, 1999; Ser. No. 60/138,893, filed Jun. 11, 1999; Ser. No. 60/142,721, filed Jul. 7, 1999; Ser. No. 60/143,185, filed Jul. 9, 1999; Ser. No. 60/153,251, filed Sep. 10, 1999; Ser. No. 60/164,633, filed Nov. 10, 1999; 60/165,813, filed Nov. 16,1999; Ser. No. 60/167,301, filed Nov. 24, 1999; Ser. No. 60/167,463, filed Nov. 24, 1999; Ser. No. 60/178,026, filed Jan. 26, 2000; Ser. No. 60/182,036, filed Feb. 11, 2000; Ser. No. 60/182,419, filed Feb. 14, 2000; Ser. No. 60/184,719, filed Feb. 24, 2000; Ser. No. 60/184,924, filed Feb. 25, 2000; Ser. No. 60/190,265, filed Mar. 17, 2000; Ser. No. 60/191,890, filed Mar. 23, 2000; Ser. No. 60/193,586, filed Mar. 30, 2000; Ser. No. 60/197,324, filed Apr. 14, 2000; Ser. No. 60/200,530, filed Apr. 27, 2000; Ser. No. 60/200,594, filed Apr. 28, 2000; and Ser. No. 60/202,087, filed May 4, 2000.

This application also incorporates by reference the following publications: K. E. van Holde, Physical Biochemistry (2^{nd} ed. 1985); William Bains, Biotechnology from A to Z (1993); Richard P. Haugland, Handbook of Fluorescent Probes and Research Chemicals (6^{th} ed. 1996); Joseph R. Lakowicz, Principles of Fluorescence Spectroscopy (2^{nd} ed. 1999); Bob Sinclair, Everything's Great When It Sits on a Chip: A Bright Future for DNA Arrays, 13 THE SCIENTIST, May 24, 1999, at 18; and Charles R. Cantor and Paul R. Schimmel, Biophysical Chemistry (1980).

### FIELD OF THE INVENTION

The invention relates to luminescence assays. More particularly, the invention relates to improvements in luminescence polarization assays by using confocal optics to increase sensitivity and accuracy, by providing additional classes of tracers, by providing improved methods to prepare specific luminescent tracers, and/or by expanding the scope of assay targets.

### BACKGROUND OF THE INVENTION

Luminescence is the absorption and subsequent re-emission of light by a luminescent molecule, or “luminophore.” In a luminescence assay, a luminophore is included in a system, and properties of the light emitted by the luminophore are used to infer properties of the system. Such light may include fluorescence and phosphorescence, and such properties may include concentration, binding, and enzymatic activity, among others.

Measurement of luminescence polarization to determine concentrations or binding is based on the principle that excitation of an anisotropic luminophore with polarized light results in emission of polarized light. The polarization will be reduced or lost if the physical orientation of the luminescent molecule changes during the lifetime of the luminophore. However, the polarization will be at least partially retained if the lifetime of the luminophore is shorter than the rotational correlation time (or “tumble time) of the molecule or complex (e.g., luminophore+binding partner) from which the luminescence is emitted. The larger the emitting moiety, the longer the tumble time, because the tumble time is proportional to the volume of the luminophore. Thus, when a small luminescent tracer binds to a larger component, the tumble time is increased, and the degree of polarization observed is enhanced. This permits a homogeneous assay whereby the binding of a labeled tracer to a moiety that enhances its size (and thus tumble time) is detected by an increase in the polarization of the luminescence emitted by the tracer. Polarization assays are described in more detail in Appendix 1.

There are a variety of formats in which this assay can be utilized. In one format, the concentration of an analyte in solution can be measured by supplying a labeled tracer that competes with the analyte for a binding moiety, especially a binding moiety larger than the labeled tracer. In this “competitive” format, the concentration of the analyte is inversely correlated with the enhancement of luminescence polarization in the light emitted by the tracer when it competitively binds the common moiety. In another format, the concentration of a target can be measured by supplying a labeled tracer that is capable of binding the target. In this case, the enhancement of polarization is a direct measure of the concentration of target. The target may further be, for example, an activated receptor, where activation can be indirectly measured by the directly measured concentration of a generated molecule or by its binding to labeled tracer per se.

Many of the labeled tracers employed in luminescence polarization assays are luminescently labeled peptides that are ligands for specific receptors or competitors to antigens for binding to antibodies. For example, U.S. Pat. No. 5,760,188 describes labeled peptide tracers for binding to the neurotensin receptor. U.S. Pat. No. 5,824,772 describes labeled peptides that bind the human somatostatin receptor. Its counterpart application, PCT Patent Application Ser. No. PCT/CA96/00207, which is incorporated herein by reference, describes, generically, receptor binding assays using peptide-labeled, luminescent tracers in a homogeneous luminescence polarization based assay. Additional such labeled peptides designed to bind to receptors are described in PCT Patent Application Ser. No. PCT/CA97/00481, which is incorporated herein by reference.

Luminescence polarization assays are important tools in life sciences research and in high-throughput screening laboratories. However, current assays suffer from a number of shortcomings. In particular, current assays involve only a limited number of tracers directed only to a limited number of targets. Thus, the use of such assays is relatively limited, especially for screening applications.

### SUMMARY OF THE INVENTION

The invention provides improvements to luminescence polarization assays by using confocal optics to increase sensitivity and accuracy, by providing additional classes of tracers, by providing improved methods to prepare specific luminescent tracers, and/or by expanding the scope of assay targets.

### BRIEF DESCRIPTION OF THE DRAWINGS

FIG. 1 shows a comparison of the results for a tyrosine kinase assay using the confocal optics/microplate of the invention as compared to conventional luminescence polarization.

FIG. 2 shows a titration plot according to the equilibrium method of the invention for determination of K_{d} characterizing the binding of labeled theophylline to a monoclonal antibody Mab-8.

FIG. 3 shows a determination of the on/off rate constants for labeled theophylline with regard to Mab-10, a different monoclonal antibody.

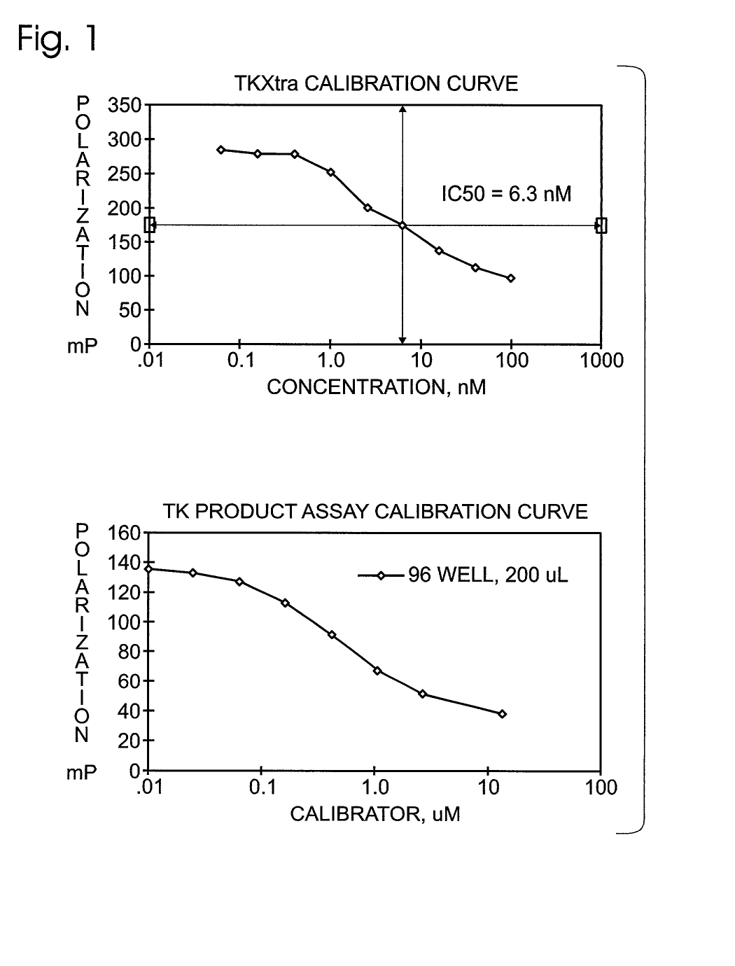

### DETAILED DESCRIPTION

The invention is directed to a multiplicity of improvements in homogeneous luminescence polarization assay techniques. The improvements may include using confocal optics to increase sensitivity and accuracy, providing additional classes of tracers, providing improved methods to prepare specific luminescent tracers, and/or expanding the scope of assay targets, among others.

In one aspect, the invention is directed to an improved method for conducting a luminescence polarization microplate assay, where the improvement comprises using confocal optics to measure the luminescence polarization of the emitted light. The sensitivity and precision of the system is thereby enhanced to a level of 3 mP (millipolarization units) standard deviation at 1 nM fluorescein, whereas in commercially available instrumentation, the standard deviation was at this level at 10 nM fluorescein.

Most of the luminescent substrates intended for luminescence polarization or other luminescence assays comprise ligands containing a luminophore. Luminophores may have a variety of emission times and can generally be classified according to whether they have a short (0.1-10 nsec) or long (10 nsec-1+sec) emission time. Here, FL-S designates a short lifetime luminophore, and FL-L a long lifetime luminophore. Typical short-lifetime luminophores include, for example, the indopyra dyes, the coumarin dyes, fluorescein, FITC, Texas red, CY-5, phycoerythrin, dansyl, rhodamine, Lucifer yellow and the like. Typical long-lifetime luminophores include metal chelates, such as the compositions described in PCT Patent Application Ser. No. PCT/US00/03589, which is incorporated herein by reference.

In general, homogeneous luminescence polarization assays involve binding of a labeled tracer to an opposite member of a specific binding pair. By “opposite member of a specific binding pair” is meant one of two components that bind to each other specifically to the exclusion of most other moieties on either side. Typical of such specific binding pairs, and most commonly thought of, are antigen/antibody interactions. However, other such interacting pairs would include, for example, receptors and their relevant ligands, biotin/avidin type interactions, complementary nucleotide sequences, triple helix forming oligonucleotides and their target duplexes, and the like. In all cases, fragments of a specific binding pair member that retain their specificity and binding affinity for the opposite member can be substituted for the complete molecule. Any member of the pair also can be coupled to solid supports or other moieties that may be useful in the context of the assays, including, of course, luminescent labels.

In another aspect, the invention is directed to a method to identify a successful tracer. In one embodiment, this is done by synthesizing candidate tracers in separate wells of a microplate and then exposing each well to a target labeled with a long-lifetime luminescent probe. Binding of the labeled target in an individual well increases the tumble time beyond the emission lifetime even of the long-lifetime luminophore, and identifies that well as containing successful tracer. The tracer thus identified can then be labeled with a suitable luminophore, either before or after cleavage from the well.

The foregoing method can also be used to identify individual tracers at various locations on a solid support, including microplate wells, when the tracers can be separated into individual components by chromatographic techniques. The method is as described in the previous paragraph and the nature of the tracer is determined from the position on the support.

In still another aspect, the invention is directed to use of luminescence polarization to determine interactions or concentrations of nonpeptide components such as oligonucleotides and other organic compounds that show sequence-specific binding, as well as methods to assay viral maturation using these assays.

It also has been possible to conduct homogeneous luminescence polarization assays in microplates. PCT Patent Application Ser. Nos. PCT/US98/14575 and PCT/US98/23095, which are incorporated herein by reference, describe a high throughput screening apparatus with a high color temperature continuous light source for such luminescence polarization and other optical assay measurements. The contents of these applications are incorporated herein by reference. The assays further may be enhanced by using microplates with elevated wells that permit assay of smaller volumes, although the reduced volume of liquid makes more important the gradient of evaporation across the plate in its effect on concentration. These plates are described in PCT Patent Application Ser. No. PCT/US98/07505, which is incorporated herein by reference. An improvement provided by the invention permits correction for the altered concentrations that may result from this gradient.

Further aspects of the invention are illustrated without limitation in the following examples:

### EXAMPLE 1

### Peptide Libraries

In one aspect, the invention is directed to methods for preparing luminescently labeled peptide tracers systematically by taking advantage of combinatorial techniques. The labeled peptide can be synthesized by parallel methodologies, such as those described by Veber, D. F. et al, Current Opinions in Chemistry and Biology (1997) 1:151-156. A multiplicity of putative tracer peptides designed for specific targets can thus be prepared in parallel by introducing the luminophore at defined positions within the peptide, for example, by synthesizing the peptides stepwise on solid phase supports and interrupting the synthesis by the insertion of a luminophore at various locations along the peptide chain. The compounds are of the formula

AAₘ-FL-AAₙ

where FL represents a luminophore, AA represents an amino acid or an analog thereof that can be accommodated into a general peptide structure, and m and n are integers such that the sum of m and n is greater than 2 but less than 200:

2<(m+n)<200

The luminophore generally may be any composition capable of luminescence emission, excluding intrinsically fluorescent naturally occurring amino acids, such as tryptophan and tyrosine (or any of the other amino acids listed in Appendix 2). However, the luminophore may include extrinsic luminophores or amino acids labeled with an extrinsic luminophore (such as fluorescein or rhodamine). The extinction coefficient, quantum yield, Stokes' shift, luminescence lifetime, and other properties of the luminophore generally may be selected according to the intended use of the labeled peptide. The parallel synthesis can be conducted on a matrix, such as in the wells of a microplate, and the putative tracer peptides retained in the designated location in the microplate well. Individual tracers, whose structures are known by reference to their location in the matrix, can then be tested with respect to the intended target, preferably using the improved methods of luminescence polarization assay described here. In addition to microplates, any addressable array is useable, such as microfabricated chips.

The result of the combinatorial synthesis is a library of peptides, which are candidates as tracers, that differ by the position of the luminophore in the peptide chain, as well as, if desired, the amino acid sequence of the chain per se. The library generally may have any number of peptides, but preferably will have a number of peptides equal to the number of wells in a microplate, or some small fraction (e.g., ½, ⅓, etc.) thereof (to account for controls, calibrators and controls, etc.). Thus, the library may have 96, 384, 1536, or more peptides, among others. Peptides that successfully bind target can be identified by enhancement of their luminescence polarization in the presence of the target. The nature of the successful tracer is established by its method of synthesis, as is understood in combinatorial synthesis methods generally.

Advantage can be taken of the availability of both FL-L and FL-S luminophores to identify suitable tracers for known targets. In this method, candidate tracers are placed into separate wells of a microplate or other suitable multicompartment or solid support arrangement. This can be done by in situ synthesis of the tracer in each array position, for example, as in the case of the combinatorial tracers whose synthesis is described above, or various fractions from a separated mixture can be placed into separate locations on a solid support or in separate wells. The separate fractions of the mixture, separated on standard chromatographic media such as DEAE cellulose, CM cellulose, phosphate-cellulose, hydroxyapatite, activated charcoal, and the like, or on chromatographic supports such as those used for thin-layer and paper chromatography, when immobilized on a solid support will have the capacity to enhance the luminescence polarization of even a long lifetime luminophore. A target coupled to a long lifetime luminophore will thus exhibit enhanced luminescence polarization if bound to the tracer immobilized on the solid support. Thus, in wells that contain tracers that can successfully bind target, there will be a diminution in the degree of luminescence polarization for the luminophore (FL-L) coupled target. Thus, wells that show such a diminution in polarization contain successful candidates.

This approach provides a straightforward method to assess a large number of tracers in relation to a defined target including those already identified by their preparation protocols, or tracers that have been fractionated into individual components. Successful tracers can then be coupled to a luminophore, preferably FL-S, either before or after decoupling from the solid support. An FL-S luminophore is preferred, as the level of polarization will then be affected most certainly by binding of the tracer to the target.

In this application, the luminescently labeled peptides that bind to target are identified by mixing a multiplicity of labeled peptides with the target, and discarding those that fail to bind. The peptides bound to target are then analyzed and synthesized afresh. In addition, PCT Patent Application Ser. No. PCT/EP97/04077, which is incorporated herein by reference, describes a method of cell sorting using assays with luminescent peptides.

### EXAMPLE 2

### Cell-Signaling Assays

In another aspect, the invention is directed to nonpeptide luminescently labeled tracers comprising a luminescent label coupled to a nonpeptide ligand or a nonpeptide product of an activated receptor. Such tracers can be used in the method of the invention to assess the effect of putative agonists or antagonists on receptors or to assess the ability of a drug candidate to affect receptor activation.

In one embodiment, a labeled ligand binds specifically to an activated form of a receptor, such as a cell-signaling receptor. The labeled ligand will exhibit a shift in luminescence polarization when bound to the activated receptor. The assay also can be constructed so as to assess the concentration of activated receptor in a sample. This can be done in the context of membrane or tissue samples or in a cellular suspension. An increase in the polarization of luminescence emitted will be correlated with the concentration of activated receptor in the sample.

Assays of this type can also be used to determine the ability of candidate drugs to affect the level of activation of the receptor. The luminescence polarization assay is run in the presence and the absence of the candidate. Drugs that interfere with the activation of the receptor will diminish the level of enhanced polarization displayed.

An example of such a labeled ligand is a stabilized, nonhydrolyzable analog of GTP, for example, GTP(γ-S) or other stabilized GTP form. GTP binds to the activated forms of a class of receptors variously known as G protein-coupled receptors, serpentine receptors, seven-span membrane receptors, and 7 transmembrane-spanning domain receptors. Upon activation, specific guanine nucleotide forms bind these receptors. GTP(γ-S) or other nonhydrolyzable form is then labeled with a luminophore such as fluorescein, such that the label still permits the activated-receptor-binding properties to be retained. Activation of the relevant receptor, such as the thrombin receptor or the various opioid receptors can then be measured in an appropriately designed assay, if desired in the presence and absence of a candidate drug.

Another example of such a labeled ligand is a product of the activation of a receptor, such as cyclic AMP (cAMP) or cyclic GMP (cGMP). Labeled forms of these secondary messengers are useful in assays to determine signaling levels by establishing competing binding for an antibody or other specific binding pair member between the labeled tracer and the cAMP or cGMP generated from the receptor activation.

The cell-signaling assays described here may be used in conjunction with compositions and techniques described in PCT Patent Application Ser. No. PCT/US00/16012, which is incorporated herein by reference.

### EXAMPLE 3

### Phosphorylation Assays

In yet another aspect, the invention is directed to specific labeled peptides containing phosphotyrosine that are useful in measuring activation of receptors characterized by phosphorylated tyrosines and to assess tyrosine phosphorylation generally. These specifically designed peptides show enhanced affinity for binding to the 4G10 antibody, which is directed to phosphorylated tyrosine. Phosphorylated tyrosine is an indicator of an activated form of the insulin receptor and many other receptors, such as EGF-receptor, and IGF-1 receptor, as well as nonreceptor tyrosine kinases, such as src, fyn, and many others known in the art. These peptides are of the formula

FL-A-pY-TGLSTRNQET-pY-ATH-NH₂;

FL-pY-pY-pY-IE-NH₂,

and

FL-G-pY-NELNLGRREE-pY-DVL-NH₂.

Here, Y is the one-letter abbreviation for tyrosine, pY is phosphorylated tyrosine, and A, T, G . . . are one-letter abbreviations for other amino acids, as specified in Appendix 2.

Preferred embodiments for FL include various FL-S moieties, especially the 5-carboxy and 6-carboxy derivatives of fluorescein, which readily form amides at the N-terminus of the peptide.

These peptides are suitable tracers for competition assays with respect to analytes that contain phosphorylated tyrosine, such as the activated insulin receptor and natural or artificial substrates of it. They are advantageous over peptide labels known in the prior art by virtue of their enhanced affinity for specific binding partners for phosphorylated peptides containing phosphotyrosines. The assay methods may be extended to phosphoserine/theronine as well.

The peptides are thus useful as tracers in any luminescence polarization assay for phosphorylated peptides where the phosphorylation is associated with tyrosine residues.

The peptides are added to a sample that contains or is suspected to contain such phosphorylated peptides along with an opposite member of a specific binding pair involving these peptides. The peptides compete with any phosphorylated protein in the sample for the opposite member so that the degree of luminescence polarization observed in light emitted from the sample is inversely correlated with the concentration of phosphorylated protein in the sample. This determination also can be made as a function of time if the activity of an enzyme that phosphorylates proteins is to be measured. In addition, the effect of compounds on such phosphorylation reactions can be studied by conducting the assay in the presence and in the absence of candidate compounds.

The phosphorylation assays described here may be used in conjunction with compositions and techniques described in PCT Patent Application Ser. No. PCT/US00/16025, which is incorporated herein by reference.

### EXAMPLE 4

### Nucleic Acid/Polymer Applications

In yet another aspect, the methods of the invention can be applied to specific binding interactions involving binding partners that are nonpeptide based, including nucleic acids and polymers containing carbohydrates. Gibson et al. Clin Chem (1997) 43:1336-41. A number of applications are envisioned.

For example, luminescently labeled tracer oligonucleotides can be used to quantify mRNA production as a measure of expression. In particular, incorporation into an mRNA molecule will enhance the luminescence polarization of the labeled oligonucleotide probe.

Similarly, genetic mutations, including deletions, insertions, or rearrangements and duplications can be assessed by shearing genomic DNA and using luminescently labeled oligonucleotides as probes. In particular, repeat sequences are of interest as the frequency of binding under controlled conditions can be compared as between nonaltered standards and genome fragments that may have additional hybridization sites or fewer hybridization sites than the standard. Very small changes, such as single nucleotide polymorphisms (SNPs), can be detected using this technique by careful control of the hybridization conditions.

Another approach to assessing mutations employs polymerase extension, which, by enlarging the molecule, enhances luminescence polarization. Polymerase extension will be interrupted by a mismatch in the tracer molecules.

In addition, binding to the class of compounds generally known as peptide-nucleic acids, which have a peptide backbone and purine or pyrimidine bases as side-chains, as described in U.S. Pat. No. 5,864,010, can be assessed. Thus, these are members of specific binding pairs useful in assays involving oligonucleotide probes labeled with luminophores. These peptide-nucleic acids themselves can be used as the labeled probes as well.

In general, the luminescence polarization methods of the invention are applicable to any class of organic compounds where sequence-specific binding is exhibited, such as calcihaemicin 1, which has sequence-specific specificity for DNA cleavage (Drak, J. et al., Proc Natl Acad Sci USA (1991) 88:7464-7468). The level of cleavage can, of course, be assessed by this technique because the size, and thus the tumble time, of the molecule is altered by such cleavage.

Another class of oligonucleotides whose affinity can be determined by the techniques of the invention comprises aptamers whose affinity for targets can be determined by on/off rates of binding to targets. U.S. Pat. No. 5,492,840 describes a flow cell surface plasmon resonance device for selecting high-affinity aptamers.

Still another application involving use of oligonucleotides is in the context of an assay for binding zinc finger proteins. Current assays for binding of zinc finger proteins to defined DNAs require gel shift analyses, which are cumbersome and only semiquantitative. The methods of the invention can be applied to such assays by coupling double-stranded DNA of about 25 base pairs with a suitable luminophore to obtain a tracer for targets containing zinc finger motifs. Increases in polarization will be observed when the DNA binding protein is present at concentrations above the K_{d} and the tracer is present at concentrations below the K_{d}. Binding may be observed within 60 minutes of incubation.

The nucleic acid/polymer assays described here may be used in conjunction with compositions and techniques described in U.S. Patent Application Ser. No. 09/494,407, which is incorporated herein by reference.

### EXAMPLE 5

### Viral Maturation

In yet another aspect, the invention is directed to application of luminescence polarization in a new context, namely, the assessment of viral maturation. Luminophores exhibiting long emission times are particularly useful in this application.

For example, an antibody that recognizes an epitope that exists only in an assembled virion but not in the isolated component proteins can be provided with an FL-L moiety to detect the titer of assembled virion as a function of time. Alternatively, an antibody that recognizes epitopes characteristic of intermediate stages of virion assembly that are no longer exposed in the completed virion can be used to monitor maturation. By adding the FL-L labeled antibody to the medium containing the assembling virions, the assembly can be followed as a change in polarization as a function of time.

Of course, by monitoring the progress of viral maturation, the effect of drugs on this process can be evaluated by conducting the experiment in the presence and absence of candidate drugs. Such drugs are believed to modulate the rate of virion assembly by interacting with the appropriate proteins involved in this process. King, J. et al. Q Rev Biol (1980) 55:369-393.

Detecting drugs that bind to immature virus particles automatically enriches for drugs that interfere with viral maturation. PCT Patent Application Ser. Nos. PCT/US98/06963 and PCT/US98/07171, which are incorporated herein by reference, describe creation of multidentate ligands with more than 10 tether points attached to a core structure of more than 10 kD; tethering, to one of these points, a luminophore with a long emission time would permit detection of binding the multidentate ligand to targets such as virions.

### EXAMPLE 6

### Affinity Constants

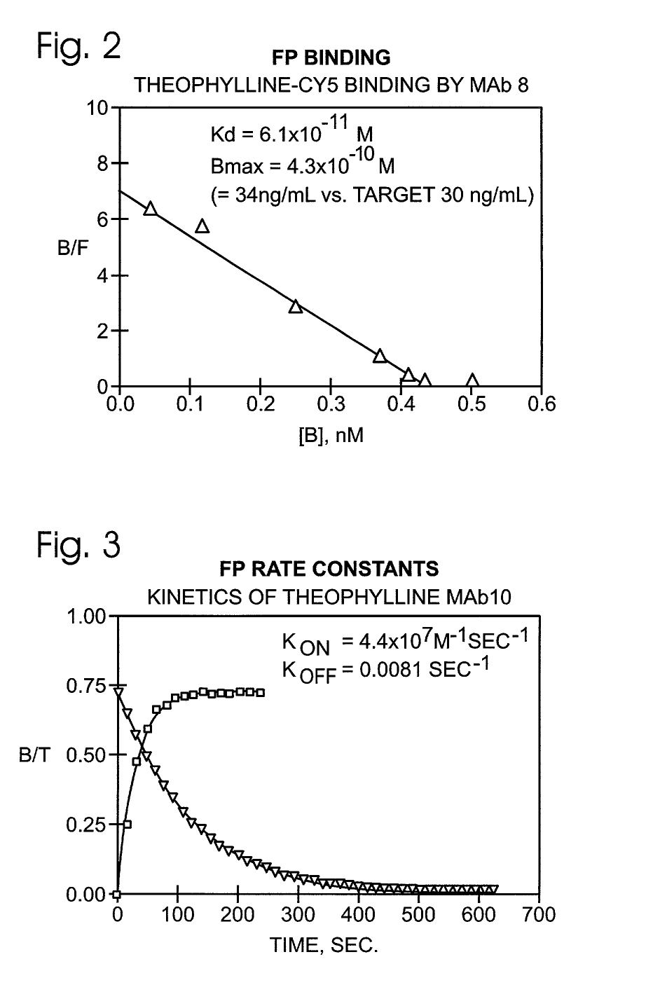

In yet another aspect, the invention may be used for determination of affinity constants for interacting molecules. In FIG. 2, an equilibrium analysis at various concentrations of labeled theophylline tracer and monoclonal antibody 8 permits calculation of the dissociation constant for these components. In FIG. 3, determination of the on/off rates of interaction of labeled theophylline with a different monoclonal antibody is illustrated. In using on/off rates, the dissociation constant is derived from a ratio of these rates. The on rate is measured by providing the components separately and measuring the rate of binding. The off rate is measured by providing the bound complex and measuring dissociation.

### EXAMPLE 7

### Volume Determinations

In yet another aspect, confocal optics may be used to judge the spatial extent of a sample, such as displacement, area, and volume. Confocal optics allows detection of light substantially exclusively from a sensed volume of a sample, as described in U.S. Pat. No. 6,071,748, which is incorporated herein by reference. This sensed volume can be used to reduce signal-to-background ratios by positioning the sensed volume away from the walls of a sample container, as described below in Example 8. This sensed volume also can be used to determine the spatial extent of a sample by positioning the sensed volume at various points along one or more axes intersecting the sample, and determining the extent based on the number and position of points from which light is collected. (The sensed volume will detect light if positioned within the sample, and will not detect light (or will detect distinguishably different light) if positioned outside the sample.) Sample extent may be determined along directions parallel and/or perpendicular to an optical axis, and may be used to determine areas and volumes by computing products of extent along two or three such directions, respectively.

This procedure also can be used to measure changes in volume. One advantage of this ability is reflected in a procedure to adjust the volume through dilution or evaporation, thus permitting alteration of concentrations in the wells. The confocal optics monitors these changes in concentration that are based on volume adjustments as opposed to addition or subtraction of analyte. The ability to adjust concentrations in this way permits titration methods using the luminescence polarization technique to define affinity constants using equilibrium characteristics. In addition, on/off rates can be used to determine dissociation constants. These measurements are independent of volume changes and merely measure binding as a function of time.

### EXAMPLE 8

### Improved-signal Assays

In yet another aspect, the invention may involve techniques for enhancing signals, signal-to-noise ratios, and/or signal-to-background ratios.

Signal may be enhanced in several ways, including (1) using a high color temperature light source, such as a xenon arc lamp, in a continuous illumination mode, (2) using a dichroic or multi-dichroic beamsplitter, and/or (3) using a sample holder whose shape is “matched” to the shape of the optical beam of the instrument, especially if the sample holder is elevated to bring the sample closer to a detector. The high color temperature light source increases the number of usable photons, which is important because the lower limit of the signal-to-noise ratio is set by the square root of the total number of photons collected in the measurement. These enhancements are described in more detail in the following U.S. Patent Applications, which are incorporated herein by reference: Ser. No. 09/349,733, Ser. No. 09/478,819, and Ser. No. 09/494,407.

Signal-to-noise ratios can be enhanced at least in part by increasing signals, for example, by using the techniques described in the previous paragraph.

Signal-to-background ratios can be enhanced in several ways, including (1) using confocal optical systems having a sensed volume to avoid luminescence from the microplate walls, (2) selecting a microplate or other substrate that increases the signal and reduces the luminescent background from materials in the microplate, (3) selecting the light sources, luminescence filters, optics, signal collection electronics, and mechanical system used in the luminescence detection optical system for maximum signal-to-background ratio, and (4) utilizing signal processing, background subtraction, and luminescence lifetime techniques, particularly FLAMe™ methodology for background reduction, as described below. These enhancements are described in more detail in the following U.S. Patent and U.S. Patent Applications, which are incorporated herein by reference: U.S. Pat. No. 6,071,748, Ser. Nos. 09/349,733, 09/478,819, and 09/494,407.

### EXAMPLE 9

### Miscellaneous Assays

Selected aspects of the invention also may be described as recited in the following numbered paragraphs:

1. A method to determine the level of expression of a gene which method comprises contacting a sample comprising the gene with a luminophore coupled to an oligonucleotide which is the opposite member of a specific binding pair of which the mRNA transcribed from the gene is the other member of the pair, and measuring the luminescence polarization light emitted from the luminophore as a measure of the expression of the gene.

2. A method to determine the level or rate of viral maturation which method comprises contacting a sample containing the virus with a luminophore coupled to a member of a specific binding pair whose opposite member is characteristic of the maturation of the virus, and measuring the luminescence polarization in the sample, optionally as a function of time, whereby an increase in luminescence polarization of the sample identifies the binding pair member as binding to a virion, the virion being the mature form of the virus.

3. A method to determine the dissociation constant of the members of a specific binding pair which method comprises measuring the rate of dissociation of members of the specific binding pair by monitoring the change in luminescence polarization of a sample containing the binding pair in coupled form, measuring the rate of association of the members of the specific binding pair by monitoring the luminescence polarization of a sample containing the members of the pair in uncoupled form, and calculating the dissociation constant as the ratio of the rates.

4. A method for determining the spatial extent of a sample positioned within a sample holder which method comprises measuring the light transmitted from a sensed volume positioned at a plurality of points within or adjacent the sample holder, and determining the spatial extent of the sample based on the presence or nature of the light transmitted from each point.

5. The method of paragraph 4, where the spatial extent is a volume.

6. The method of paragraph 4 further comprising repeating the steps of measuring and determining at two different times, and calculating the change in spatial extent between the two different times.

7. The method of paragraph 6, wherein the change in spatial extent represents a dimunition in volume.

8. The method of paragraph 4, wherein the sample holder is a microplate.

9. The method of paragraph 8 further comprising the steps of measuring and determining for at least two wells in the microplate.

Thus, the invention provides a variety of improvements and additional applications for luminescence polarization assays.

Although the invention has been disclosed in its preferred forms, the specific embodiments thereof as disclosed and illustrated herein are not to be considered in a limiting sense, because numerous variations are possible. As used herein, singular terms do not preclude the use of more than one of the associated element, and embodiments using more than one of a particular element are within the spirit and scope of the invention. Applicants regard the subject matter of their invention to include all novel and nonobvious combinations and subcombinations of the various elements, features, functions, and/or properties disclosed herein. No single feature, function, element or property of the disclosed embodiments is essential. The following claims define certain combinations and subcombinations of features, functions, elements, and/or properties that are regarded as novel and nonobvious. Other combinations and subcombinations may be claimed through amendment of the present claims or presentation of new claims in this or a related application. Such claims, whether they are broader, narrower, equal, or different in scope to the original claims, also are regarded as included within the subject matter of applicants' invention.

### Appendix 1

### Luminescence Polarization Assays

Luminescence polarization assays involve the absorption and emission of polarized light. (Polarization describes the direction of light's electric field, which generally is perpendicular to the direction of light's propagation.) In a luminescence polarization assay, specific molecules within a composition are labeled with one or more luminescent moieties (i.e., “luminophores”). The composition then is illuminated with polarized excitation light, which preferentially excites luminophores having absorption dipoles aligned parallel to the polarization of the excitation light. These molecules subsequently decay by preferentially emitting light polarized parallel to their emission dipoles. The extent of polarization of the total emitted light depends on the extent of molecular reorientation during the time interval between luminescence excitation and emission, which is termed the luminescence lifetime, τ. In turn, the extent of molecular reorientation depends on the luminescence lifetime and the size, shape, and environment of the reorienting molecule. Thus, luminescence polarization assays may be used to quantify binding reactions and enzymatic activity, among other applications. In particular, molecules commonly rotate (or “tumble”) via diffusion with a rotational correlation time τᵣₒₜ that is proportional to their volume, or the cube of their radius of gyration. (This cubic dependence on radius makes polarization assays very sensitive to binding.) Thus, during their luminescence lifetime, relatively large molecules will not reorient significantly, so that their total luminescence will be relatively polarized. In contrast, during the same time interval, relatively small molecules will reorient significantly, so that their total luminescence will be relatively unpolarized.

The relationship between polarization and intensity is expressed by the following equation:

$$P = \frac{{I}_{\vert \vert} - I ⊥}{{I}_{\vert \vert} + {I}_{⊥}} \qquad ( A1 )$$

Here, P is the polarization, I_{∥} is the intensity of luminescence polarized parallel to the polarization of the excitation light, and I_{⊥} is the intensity of luminescence polarized perpendicular to the polarization of the excitation light. P generally varies from zero to one-half for randomly oriented molecules (and zero and one for aligned molecules). If there is little rotation between excitation and emission, I_{∥} will be relatively large, I_{⊥} will be relatively small, and P will be close to one-half. (P may be less than one-half even if there is no rotation; for example, P will be less than one-half if the absorption and emission dipoles are not parallel.) In contrast, if there is significant rotation between absorption and emission, I_{∥} will be comparable to I_{⊥} and P will be close to zero. Polarization often is reported in milli-P units (1000×P), which for randomly oriented molecules will range between 0 and 500, because P will range between zero and one-half.

Polarization also may be described using other equivalent quantities, such as anisotropy. The relationship between anisotropy and intensity is expressed by the following equation:

$$r = \frac{{I}_{\vert \vert} - {I}_{⊥}}{{I}_{\vert \vert} + 2 {I}_{⊥}} \qquad ( A2 )$$

Here, r is the anisotropy. Polarization and anisotropy include the same information, although anisotropy may be more simply expressed for systems containing more than one luminophore. In the description and claims that follow, these terms may be used interchangeably, and a generic reference to one should be understood to imply a generic reference to the other.

The relationship between polarization and rotation is expressed by the Perrin equation:

$$( \frac{1}{P} - \frac{1}{3} ) = ( \frac{1}{{P}_{0}} - \frac{1}{3} ) \cdot ( 1 + \frac{τ}{{τ}_{rot}} ) \qquad ( A3 )$$

Here, P₀ is the polarization in the absence of molecular motion (intrinsic polarization), τ is the luminescence lifetime (inverse decay rate) as described above, and τᵣₒₜ is the rotational correlation time (inverse rotational rate) as described above.

The Perrin equation shows that luminescence polarization assays are most sensitive when the luminescence lifetime and the rotational correlation time are similar. Rotational correlation time is proportional to molecular weight, increasing by about 1 nanosecond for each 2,400 dalton increase in molecular weight (for a spherical molecule). For shorter lifetime luminophores, such as fluorescein, which has a luminescence lifetime of roughly 4 nanoseconds, luminescence polarization assays are most sensitive for molecular weights less than about 40,000 daltons. For longer lifetime probes, such as Ru(bpy)₂dcbpy (ruthenium 2,2′-dibipyridyl 4,4′-dicarboxyl-2,2′-bipyridine), which has a lifetime of roughly 400 nanoseconds, luminescence polarization assays are most sensitive for molecular weights between about 70,000 daltons and 4,000,000 daltons.

We claim:

1. A combinatorial method to prepare a library of peptide tracers for use in a luminescence-based assay having the formula
AAₘ—FL—AAₙ,
wherein each AA is independently an amino acid, FL is a luminophore different than a naturally occurring amino acid, and m and n are integers such that the sum of m and n is greater than 2 but less than 200, which method comprises
synthesizing the peptide tracers stepwise on solid phase supports through the addition of an AA or FL, wherein FL is inserted at the same or different position, thereby varying or not the values of m and n and the type of AA selected so as to obtain a library of labeled peptides tracers.

2. A library of labeled peptides synthesized by the method of claim 1.

3. The library of claim 2, wherein the number of labeled peptides in the library is at least 96.

4. The library of claim 3, wherein the number of labeled peptides in the library is at least 384.

5. The method of claim 1, wherein the peptide tracers are prepared in parallel.

6. The method of claim 1, wherein the solid phase supports are wells of a microplate.

7. The method of claim 1, further comprising
cleaving the peptide tracers from the solid phase supports.

8. The method of claim 1, the luminescence-based assay involving binding between at least one of the peptide tracers and a target, further comprising
identifying any peptide tracers that bind to the target.

9. The method of claim 8, wherein the step of identifying any peptide tracers that bind includes the steps of mixing a multiplicity of labeled peptide tracers with the target.

10. The method of claim 8, wherein the step of identifying any peptide tracers that bind includes the steps of
exposing the solid phase supports to a target labeled with a long-lifetime emission luminophore (FL-L),
subjecting the solid phase support to polarized light comprising a wavelength for excitation of the FL-L so as to effect emission of polarized light,
measuring the degree of polarization of the emitted light at each defined location on the solid support, and
identifying as a peptide tracer useful in luminescence polarization assays for the target, a peptide tracer at a solid phase support having a low degree of measured polarization.

11. The method of claim 10, further comprising
labeling the identified successful peptide tracer with a short-lifetime luminophore before or after cleaving the tracer from the solid phase support.

12. A library of luminescent peptide tracers comprising a multiplicity of peptides of the formula
AAₘ-FL-AAₙ,
wherein each AA is independently an amino acid, FL is a luminophore different than a naturally occurring amino acid, and m and n are integers such that the sum of m and n is greater than 2 but less than 200, and wherein the values for m and n differ among the individual peptide members of the library.

13. The library of claim 12, wherein the number of labeled peptides in the library is at least 96.

14. The library of claim 13, wherein the number of labeled peptides in the library is at least 384.

15. A method to identify a tracer for use in a luminescence polarization assay for a target which tracer is the opposite member of a specific binding pair comprising the target which method comprises
placing candidate tracers at separate, defined locations on a solid support,
exposing the locations on the support to a target labeled with a long-lifetime emission luminophore (FL-L),
subjecting the solid support to polarized light comprising a wavelength for excitation of the FL-L so as to effect emission of polarized light,
measuring the degree of polarization of the emitted light at each defined location on the solid support, and
identifying as a tracer useful in luminescence polarization assays for the target, a tracer at a defined location having a low degree of measured polarization.

16. The method of claim 15 further comprising
labeling the identified successful tracer with a short-lifetime luminophore before or after cleaving the tracer from the solid support.

17. The method of claim 15, wherein the locations on solid support are wells of a microplate.

18. The method of claim 15, wherein the displaying of candidate tracers is accomplished by synthesis of the tracers at the various locations on the solid support.
